(19) United States
(12) Patent Application Publication
(10) Pub. No.: US 2010/0179557 A1
(43) Pub. Date: Jul. 15, 2010

(54) Adjustable Powered Rongeur

(76) Inventor: Daniel S. Husted, Stuart, FL (US)

Correspondence Address: BARLEY SNYDER, LLC, 1000 WESTLAKES DRIVE, SUITE 275, BERWYN, PA, 19312, US

(21) Appl. No.: 12/354,497

(22) Filed: Jan. 15, 2009

(51) Int. Cl. A61B 17/00; A61B 5/00

(52) U.S. Cl. 606/83; 600/300

(57) ABSTRACT

The invention relates to a powered medical instrument having an adaptable deburring bit and independent nerve sensors that facilitate positioning of the instrument to a proximate surgery site. The medical instrument having a hand piece on a proximal end of a shaft and a hollow tip portion on a distal end of the shaft. The hand piece includes a handgrip and a squeezable trigger portion, whereby the trigger portion is independently compressible of the handgrip. The trigger portion controlling a rotatable surgical tool bit, which is housed in the tip portion and powered by a connecting drive system. The medical instrument further includes a safety apparatus provided on the grip portion, capable of locking the instrument and a monitoring system disposed on the tip portion, in order to identify proximity of nerve endings.

### FIELD OF THE INVENTION

The invention relates to a powered medical instrument, in particular, the invention relates to a powered medical instrument having an adaptable deburring bit and independent nerve sensors that facilitate positioning of the instrument to a proximate surgery site.

### BACKGROUND

A conventional rongeur is a medical device constructed of a sharp-edged, scoop-shaped tip, used for gouging out bone. The rongeur is designed to chip away, shave, or gnaw bone fragments or tissue through a cutting device, commonly on a distal end of the instrument.

Rongeurs are made in a variety of sizes and shapes, designed for different applications, consistent with their intended purposes. A rongeur designed to remove bone is generally quite strong, usually made of surgical steel, and designed to withstand a great amount of force applied to the bone.

As is well known in the medical community, a Kerrison type rongeur is utilized in certain types of spinal and back operations. The Kerrison type rongeur is designed to cut bone near the spinal column, commonly designed to have an elongated scissor type component including a pair of small end plates that are spaced from each other, yet movable relative to the other, as well as a trigger-like device that moves one of the end plates relative to the other. Many of the common Kerrison type rongeurs employ a manual snipping action and are not power driven.

The Kerrison type rongeur works by allowing the surgeon to place the instrument into an affected region, and then proceed to cut bone. For instance, in a foraminotomy, the affected region is generally between a compressing bone and an underlying nerve area, the nerve being compressed and causing a patient pain. The surgery involves the removal of the compressing bone, using a Kerrison type rongeur to extract or remove pieces of that imperfect bone. Thereby, the surgical procedure relieves compression exerted on the nerve.

As in many situations, every procedure and surgery site is atypical. The area may be crowded or extra-sensitive, and therefore not be conducive to use a conventional Kerrison type rongeur. Modification of medical instruments, such as rongeurs, is common practice. Surgeons and medical device companies continually modify the traditional rongeur to better suit surgical procedures. For instance, the traditional rongeur was a straight design and gouged bones with powerful grips. However, surgeons modified the traditional rongeur to have curved housings and drill bits, rather than the conventional rongeur that is straight and gouges at the bone.

For example, U.S. Pat. No. 4,586,497 discloses a drill fixation device adapted to fit a high-speed drill, which is then used during spinal surgery for drilling and cutting of bone vertebra. The device comprises a base, a relatively small housing that connects to an existing drill, and a squeeze grip that provides engagement of a rotating drill bit with vertebra bone. Further, a footplate connects to the base by a long shaft to be in the path of the drill bit. In use, the footplate is positioned beneath the bone to be cut and applies counterforce to the bone material as the drill cuts, yet shields nerve tissue from damage by the bit. The intensity of the drilling depends on the amount of force applied to the squeeze grip. While the '497 patent discloses a rongeur type power drill with protective footplate and handled trigger, that device is rather bulky and creates the potential to damage a nerve, not protected by the footplate. Further, the trigger lacks any ability to control drill bit speed, adjustability and precision. The trigger is only used to create pressure between the target area (bone) and the drill bit, and may be very difficult to precisely position.

U.S. Patent Application Publication No. 2005/0165420 A1 discloses a powered rotatable surgical deburring tool, as well, which comprises a protective housing, a dissecting footplate, and a powered deburring bit all of which as said to provide enablement of various spinal decompression procedures. The instrument further comprises a hand piece, a rigid straight shaft portion, which extends from the hand piece and has a distal end and a proximal end. Depending on the embodiment, the rigid shaft portion then connects to either an outer tube via a pivot joint or a flexible shaft.

A drive shaft is located within the shaft portion and extends through the distal end of the outer tube or flexible shaft. This drive shaft facilitates the rotation of the deburring bit, which sits within the protective housing and is only partially exposed. The protective housing can rotate relative to the longitudinal axis of the deburring bit and is designed to protect nerve roots from the deburring bit. Although the publication discloses a rotatable surgical deburring tool, which facilitates surgical tool placement, the reference is silent on avoiding inadvertent damage in the surgery site. Overall, the device is silent on bit adjustment, modification, and replacement, making it difficult to position for many types of surgeries, including spinal and back surgery.

Back and spinal surgery is delicate and involves many risks, especially considering that the surgery occurs so close to the actual spinal cord and other sensitive nerve endings. Therefore, the multiple nerve endings make it a very delicate procedure. A voluntary or involuntary erroneous move by a surgeon can cause nerve damage and even paralysis. The risk of nerve damage and paralysis following spinal and back surgery presents a serious problem for surgeons is avoiding. The small nerve endings may be inadvertently damaged by even the most experienced surgeon. Often times, just trying to identify the nerves can stretch or tear the nerve resulting in irreparable damage. Consequently, nerve-monitoring techniques have been developed to facilitate location of nerve endings during surgery, and improved instrument positioning.

For example, U.S. Pat. No. 5,928,158 discloses a powered surgical instrument used for cutting tissue with an electronic nerve sensor attached to the tip of the device. The surgical instrument comprises a housing, a cutting device found on the distal end of the housing, and a sensor, which alarms the user of a proximate nerve root by means of an LED or speaker. In order for the sensor to detect a nerve root, an electrical signal is communicated to the patient through an electrical lead and an electrical patch. The patch attaches to the patient and emits an electrical signal through the patient's nervous system. If the sensor picks up the signal from a proximate nerve, then the surgeon is alarmed. The patent discloses several embodiments, varying both the type of cutting device and sensor used. Although the '158 patent discloses a surgical instrument used for cutting tissue having an electronic sensor in order to detect nerve roots, the patent is silent on bit adjustment, modification, and replacement, making it difficult to position for many types of surgeries, including spinal and back surgery.

### SUMMARY

In light of the shortcomings of the prior art, and long felt need for precision instruments, the invention provides a powered medical instrument having an adaptable deburring bit and independent nerve sensors that facilitate positioning of the instrument to a proximate surgery site.

The medical instrument has a hand piece on a proximal end of a shaft and a hollow tip portion on a distal end of the shaft. The hand piece includes a handgrip and a squeezable trigger portion, whereby the trigger portion is independently compressible for the handgrip. The trigger portion controls a rotatable surgical tool bit, which is housed in the tip portion and powered by a connecting drive system. The medical instrument further includes a safety apparatus provided on the grip portion, capable of locking the instrument and a monitoring system disposed on the tip portion, in order to identify proximity of nerve endings.

A related medical instrument monitoring system has two or more sensors positioned on sides of the medical instrument's distal end, each sensor attaching to an external CPU through conductors, the external CPU being used to identify signals transferred between a nerve and the sensor.

### BRIEF DESCRIPTION OF THE DRAWINGS

The invention will be explained in greater detail in the following with reference to embodiments, referring to the appended drawings, in which:

FIG. 1 is a side view of a first embodiment of the invention;

FIG. 2 is a close-up sectional view of the first embodiment of the invention, focusing on a hollow housing tip portion;

FIG. 3 is a front view of the first embodiment of the invention;

FIG. 4 is a top view of the first embodiment of the invention;

FIG. 5 is a side view of a second embodiment of the invention;

FIG. 6 is a close-up sectional view of the second embodiment of the invention, focusing on a hollow tip portion;

FIG. 7 is a close-up top view of the second embodiment of the invention, focusing on a safety apparatus.

### DETAILED DESCRIPTION OF THE EMBODIMENT(S)

The invention will now be described in greater detail. Referring first to FIG. 1, a surgical instrument 1 is shown, having a shaft 6 connecting a hand piece 2 and a hollow tip portion 4. The hand piece 2 is positioned on a proximal end of the shaft 6, while the tip portion 4 is positioned on a distal end of the shaft 6. In the embodiment shown, the shaft 6 and tip portion 4 are designed in such a way that the surgical instrument 1 is elongated and slightly curved, in accordance with a traditional design of a Kerrison type rongeur.

A handgrip 10 and a squeezable trigger section 20 make up the hand piece 2 of the surgical instrument 1. The handgrip 10, elongated and shaped as shown in FIG. 1, is constructed integrally with the elongated shaft 6 section of the surgical instrument 1.

The trigger section 20 includes a first trigger 22 and a second trigger 24, both connecting to the surgical instrument 1. However, it is possible to design the invention having a trigger section 20 with one or more triggers. The first trigger 22 is pivotally connected at a point where the shaft 6 and the handgrip 10 meet. As shown, the first trigger 22 attaches to the surgical instrument 1 using a first locking pin 26, passing through a hole in the proximal end of the first trigger 22 and corresponding holes formed through the surgical instrument 1.

The second trigger 24 is attached to the surgical instrument 1 using the same first locking pin 26. However, a hole is formed substantially in a middle portion of the second trigger 24, where the proximal end of the second trigger 24 extends through an opening in the underside of the surgical instrument 1. It is possible to fasten either trigger 22, 24 using a variety of fastening means, such as a pivot pin, bolt and nut, etc. One skilled in the art will appreciate that either trigger 22, 24 can be attached in various ways, as long as the modification does not depart from the scope and intended purpose of the invention as disclosed in the accompanying claims.

The handgrip 10 is designed to fit in a user's palm, allowing the user's fingers to operate the first and second triggers 22, 24. In the embodiment shown, the handgrip 10 is a hollow unitary extension of the shaft 6. However, it is possible to provide a handgrip 10 that is a separate, non-unitary component, connecting to shaft 6, by any suitable means.

The ergonomic shape of the handgrip 10 and placement of the first trigger 22 and second trigger 24 allow the one-handed use of the surgical instrument 1. Other ergonomic shapes of the handgrip 10 and other shapes/placements of the associated components are possible without limiting the scope or intent of the present invention.

A spring 28, prepared from a coiled piece of metal wire, attaches to both the first trigger 22 and the handgrip 10. The spring 28 resiliently applies tension to the squeezable first trigger 22, since the handgrip 10 is stationary. A traditional leaf spring or other types of springs could be used here as well.

According to a first embodiment of the present invention, FIG. 1 shows a safety apparatus 12 provided on the rear side of the handgrip 10, internally connecting to a drive system 80. It should be noted that an illustration of the drive system 80 is shown in abstract, since the drive system 80 may be one of many drive systems known to the medical arts, including but not limited to the drive system described in U.S. Patent Application Publication No. 2005/0165420 A1.

In the present embodiment, the safety apparatus 12 is an electric switch connecting to the drive system 80. However, it is possible to mechanically connect the safety apparatus 12 to the first and second triggers 22, 24, through the handgrip 10. The safety apparatus 12 is positioned on the handgrip 10 in such a way that the safety apparatus 12 may be functioned by use of a free thumb, while having the palm rest on the handgrip 10. It is possible, however, to include the safety apparatus in other areas of the surgical instrument 1, which may be convenient for one hand use of the surgical instrument 1, as well.

The tip portion 4 is pivotally connected to the shaft 6 at a pivot point 30, using a second locking pin 29. The tip portion 4 also includes a rotating deburring bit 40 housed on the inside of the tip portion 4, while a monitoring system 50 is attached to the outer surface of the tip portion 4. The deburring bit 4 sits within the hollow tip portion 4 in such a way that the deburring bit is substantially enclosed within the tip portion 4.

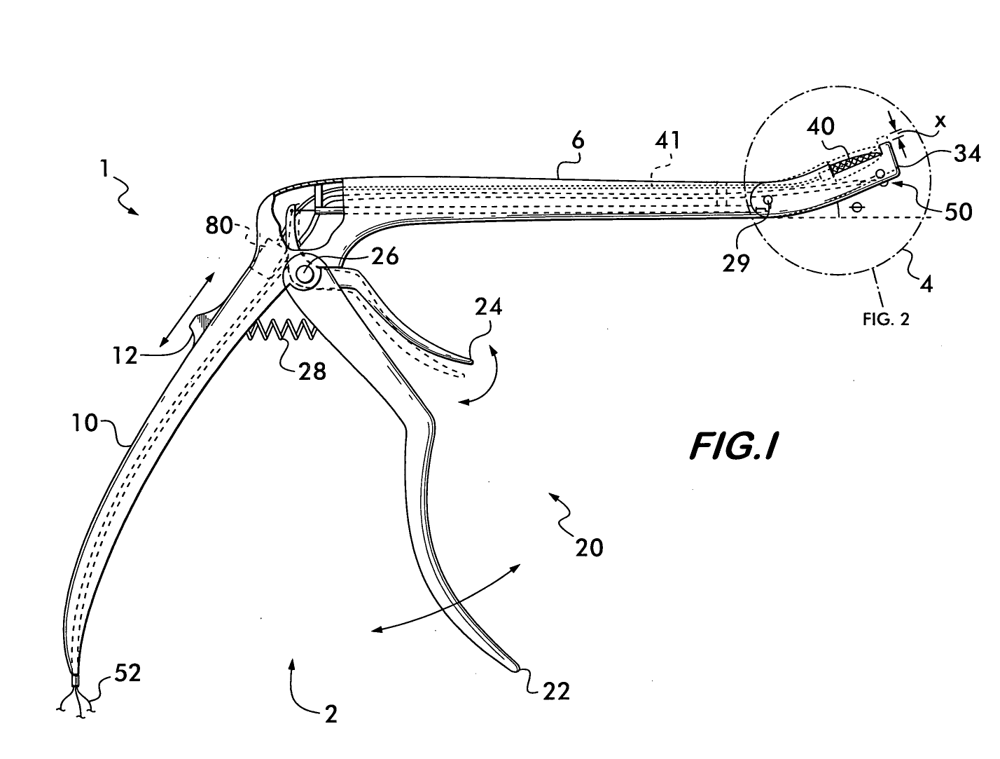

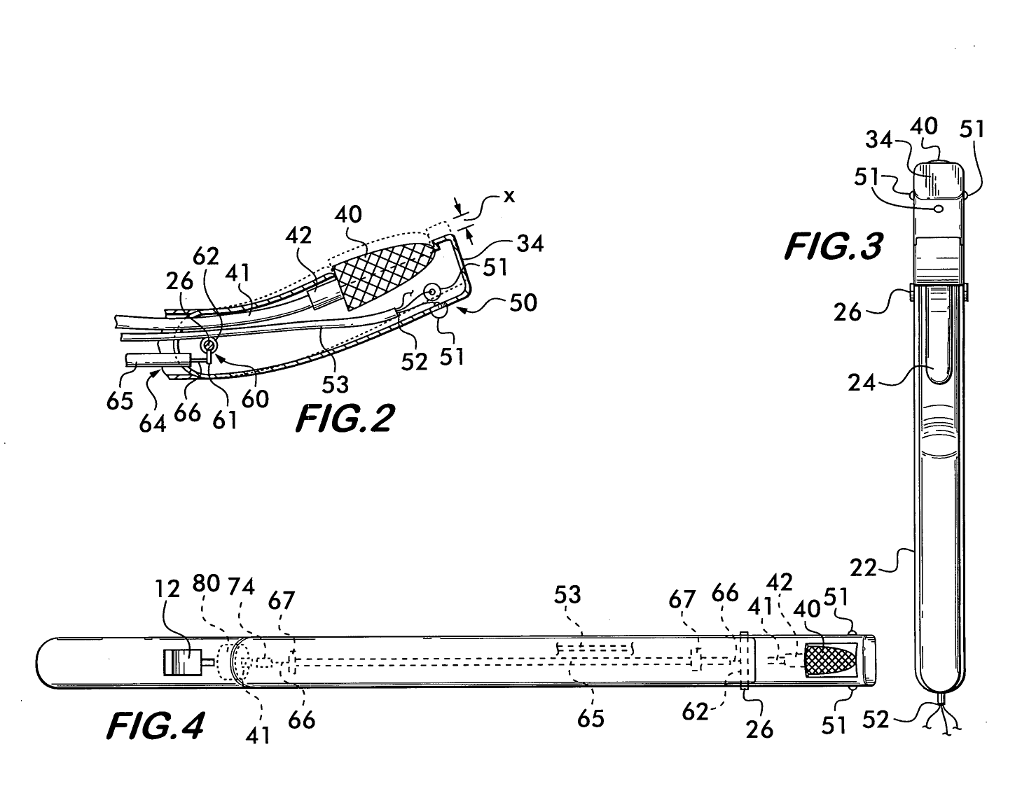

FIG. 2 shows a close-up section view of the tip portion 4, according to the first embodiment of the invention. The tip portion 4 is designed as a unitary component of the surgical instrument 1. The tip portion 4 has a pivot mechanism 60 suitably fastened between each inner side of tip portion 4. The pivot mechanism 60 includes a hollow axel rod 62, which allows the second locking pin 29 to run through the center and connect the tip portion 4 and the shaft 6. A lever 61 is rigidly attached to the underside of the axel rod 62 at one end, and further connecting to a telescoping actuator 64 at the other end. The telescoping actuator 64, prepared from a central line 66 and a rigid protective sleeve 65, runs through the entire length of the shaft 6 and connects to the proximal end of the second trigger 24 (as shown in FIG. 1). It is also possible to prepare the telescoping actuator 64 without the protective sleeve 65. However, the protective sleeve 65 connects to the shaft 6, providing further rigidity, as will be discussed below

The tip portion 4 includes two holes prepared where the hollow axel rod 62 connects to the inner surface of the tip portion 4, creating an opening to be received by a fastening mechanism, such as the second locking pin 29. The distal end of the shaft 6 is designed to receive the proximal end of tip portion 4, and has two holes arranged to match the two holes formed on the tip portion 4. The second locking pin 29 runs through each formed hole, as well as the hollow axel rod 62, connecting the tip portion 4 and shaft 6.

As discussed above, the tip portion 4 may be prepared as a unitary extension of the shaft 6. When both the tip portion 4 and the shaft 6 are connected, regardless of construction design, their union defines an angle θ, which is adjustable in the present embodiment. Such a construction lends to the traditional design of a Kerrison type rongeur. In a unitary construction, where the tip portion 4 is an extension of the shaft 6, the angle θ would be fixed, and the housing design would be prepared according to manufacturing and surgical accommodation.

As shown, the tip portion 4, substantially hollow in design, includes a blunt faceplate 34. A substantial area of the tip portion's 4 top surface is left open, having the deburring bit 40 extend ever so slightly through the top surface, by a height x. In the present embodiment, the bit's top surface is approximately 1 mm above the top planar surface of the tip portion 4, exposing the bit 40 for contact at the affected surgical site. However, it is possible to have the bit 40 extending higher or lower from the surface of the tip portion 4, which may depend on surgical procedure or preference.

In the embodiment shown, the deburring bit 40 is designed in the shape of a bullet, with teeth made of carbide, tungsten carbide, etc., for precise, efficient grinding and cutting. Although the bit 40 is replaceable, the present invention provides many options to adjust the bit 40 performance such that the surgeon may only need to replace or substitute the bit 40 during infrequent occurrences, such as wear. Overall size and bit 40 dimensions can be modified according to surgical accommodation, as can the overall size and tip portion 4 dimensions.

The deburring bit 40 connects to the internal drive system 80 through a drive shaft 41. The drive system 80 controls the bit 40 rate of rotation. Although the current embodiment provides a drive system 80 powered by electricity, a pneumatic or other powered drive system 80 custom designed for surgical tools can be implemented. As discussed above, drive systems are well known in the industry, and a drive system like the one disclosed in U.S. Patent Application Publication No. 2005/0165420 A1 would be most acceptable.

As shown in FIG. 1, the drive system 80, such as an electrical motor, may be integrally positioned within the surgical instrument 1. The first trigger 22 connects to the drive system 80 through wiring, while the deburring bit connects to the drive system 80 through a drive shaft 41. In the present embodiment, the drive system 80 is internally positioned within the hollow portion of the shaft 6, as illustrated. However, the drive system 80 may be designed as an external component to facilitate easier and more efficient sterilization of the surgical instrument 1.

The drive shaft 41 is flexible, accommodating the curved design of the surgical instrument 1. Additionally, the drive shaft 41 may run through the entire instrument 1, especially if the drive system 80 is positioned externally from the instrument 1. In the present embodiment, and shown in FIG. 2, the drive shaft 41 includes a rigid protective sheathing 42 at the distal end of the drive shaft 41. The protective sheathing 42 is fixed, possibly by a weld, to the inner surface of the tip portion 4, in order to stabilize the rotatable deburring bit 40. The protective sheathing 42 may also run the entire length of the drive shaft 41 as well, which would be used to provide drive shaft 41 protection from certain impurities surrounding the surgical site.

In the present embodiment, the deburring bit 40 fastens to the drive shaft 41 through a screwing means, where a screw is prepared on the proximal end of the bit 40, and a thread on the distal end of the drive shaft 41. The fastening is performed by screwing the bit 40 onto the drive shaft 41 in a direction opposite the rate of bit 40 rotation. However, it is possible to connect the bit 40 in a variety of fastening means known to the art.

The tip portion 4, although hollow, is constructed quite rigid and capable of holding to form, even under extreme pressure and heat. In the present embodiment, the tip portion 4 would be made from the surgical material, such as surgical steel.

FIGS. 2 through 4 illustrate a nerve monitoring system 50 applied and adapted to the surgical instrument 1. The monitoring system 50 is made up of sensors 51 found on the distal end of the tip portion 4. Each sensor 51 separately connects to an external CPU system (not shown) using conductors 52 that run internally through the surgical instrument 1, where the conductors 52 come out of the distal end of the handgrip 10. Sheathing 53 may be applied to protect the conductors 52 from external impurities, as well as extreme elements encountered during sterilization.

In the embodiment shown, two sensors 51 are placed on each side of tip portion 4, while another sensor 51 is positioned on the underside of the tip portion 4. Each sensor 51 is positioned on the outer surface of the tip portion 4, however, it is possible to prepare the tip portion 4 with integrated sensors 51.

FIG. 4 is a top view of the surgical instrument 1, illustrating how the conductors 52, telescoping actuator 64, and flexible drive shaft 41 run through the hollow shaft 6. The telescoping actuator 64, specifically the protective sleeve 65, connect to shaft 6 at connection points 67, rigidly attaching the protective sleeve 65. The conductors 52, like the drive shaft 41, are flexible. In the embodiment shown, the protective sheathing 42 is prepared rigid in area where the drive shaft 41 attached to the bit, in order to maintain precise positioning of the bit 40.

Referring back to FIG. 1, the first trigger 22 is curved and designed for use by the lower fingers. The first trigger 22 should be substantially longer than second trigger 24. The length of the first trigger 22 promotes better range of operation and freedom, as well as greater instrument 1 control.

The first trigger 22 connects to the internal drive system 80. The internal drive system 80 further connects to an external power source through wires, and as discussed above, the power source can be electric, pneumatic, etc.

The second trigger 24 is designed to be shorter than the first trigger 22, and has the proximal end long enough to extend into the inner surface of the surgical instrument 1, as is illustrated quite clearly in FIG. 1. The second trigger 24 is further designed to accommodate use by the index finger. The distal end of the second trigger 24 should extend far enough from the first trigger 22, so that neither trigger 22, 24 can disrupt the function of the other. As shown, the second trigger 24 extends in a direction away from first locking pin 26, toward the tip portion 4, and away from the first trigger 22 in a direction toward the instrument shaft 6. As discussed above, the second trigger 24 connects to the pivot mechanism 60 through the telescoping actuator 64. The central line 66 of the telescoping actuator 64, connects to the proximal end of the second trigger 22, which extends into the surgical instrument 1.

FIGS. 1 and 4 illustrate the position of the safety apparatus 12 of the first embodiment. The safety apparatus 12, being a switch, designed for use by the thumb. As discussed above, the safety apparatus 12 electrically connects to the drive system 80.

Hereinafter, descriptions will be given to the function of the first embodiment of the present invention.

Prior to operation, the safety apparatus 12 locks the surgical instrument 1, negating connection between the power source and the drive system 80, essentially negating any rotation of the deburring bit 40. Therefore, the user can position the tip portion 4 into an affected region, before the user proceeds with bit 40 rotation, without the unintentionally affecting potential surrounding nerve endings.

To improve the safety of the instrument 1 placement the monitoring system 50, using nerve sensors 51 and an external CPU system (not shown), identifies proximity of potential nerve endings in the surgical site.

Each sensor 51 picks up a signal communicated through the patient's local nervous system, by leads (not shown) placed on a specific region of the patient's body. The signal, sent by an external CPU system (not shown), loops back through the human nervous system and received by the sensor 51. The sensor 51 sends the signal back to the CPU system, causing a type of circuit. As a result, the user can identify the proximity of nerve endings by monitoring which sensors 51 pick up signals. The strength of a signal can be monitored as well, further identifying the proximity of any nerve endings.

Each signal is received by a sensor 51 when a nerve comes into close proximity with the sensor 51. The user can calibrate what this distance will be, before he starts the operation. If the external monitoring system (not shown) displays that the left side sensor 51 receives a signal, then the user knows to avoid that site, for fear of damaging the nerve during the procedure. Having a sensors 51 located on both sides of the tip portion 4, as well as a sensor 51 on the underside tip portion 4, allows the user to avoid nerve endings. Further, the sensor 51 on the underside of the tip portion 4, allows the user to position the deburring bit 40 opposite the nerve ending position (see FIG. 3).

The sensors 51 in the present embodiment receive signals. However, it is possible to design the monitoring system 50 where the sensors 51 transmit signals to proximate nerves. For instance, each sensor 51 would send out a different signal, and the surgeon would monitor patient response to those sent signals. Such a response maybe a physical response, i.e. twitch, or an electronic response, i.e. reverse circuit described above.

Depending on the type of observed signals, the user can identify the proximate position of nerve endings in the surgical site, and avoid temporary or permanent damage of those nerves.

To further improve on positioning of the instrument 1 within the surgery site, the second trigger 24 can position the bit 40 by the angle θ. As the user squeezes on the second trigger 24, the second trigger 24 causes the telescoping actuator 64 to push against the pivot mechanism 60, increasing the angle θ between the shaft 6 and the tip portion 4. Increasing the angle θ causes the tip portion 4 and the deburring bit 40, housed in the tip portion 4, to become more inclined. If the user pushes the second trigger 24 in a direction away from the first trigger 22, then the tip portion 4 and deburring bit 40 will become less inclined, resulting in a flatter position.

As a result of bit 40 adjustment, the instrument 1 has better range of motion and can be precisely positioned within the surgery site. The angle 0 should accommodate surgery type and position tip position desired by the user. The more angle 0, the deburring bit will perform more aggressive cutting. Further, the ability to move the tip portion 4 in a variety of angles θ improves on the freedom of bit placement. Hence, the user can limit or even damage to unaffected areas. Overall, the second trigger 24 would be designed to only position the tip portion 4 approximately 1-3 mm higher from the starting position. However, it is possible to provide more or less degree of motion.

Since the second trigger 24 can move, even during operation, the fluidity and movement of the second trigger 24 may be adjusted by the user. Therefore, the user could adjust the fastening of the second trigger 24 to be looser or tighter, making movement of the second trigger 24 less or more fluid. This would apply to the first trigger 22, as well.

Once the user determines the appropriate instrument 1 position, the user would unlock the safety apparatus 12 by simply sliding the safety apparatus 12 to an unlocked position with a free thumb. In an unlocked position, the safety apparatus 12 allows power to flow to the drive system 80, thus activating the drive system 80 and facilitating rotation of the bit 40. When the safety apparatus 12 is in the locked position, no power is allocated to the drive system 80, and the bit 40 cannot rotate.

The first trigger 22 controls rate of bit 40 rotation. As the user squeezes the first trigger 22 toward the handgrip 10, the bit 40 starts its rotation. In fact, in the present embodiment, the bit 40 rotation accelerates ever increasing with greater compression. A transducer (not shown) may be used to identify the amount of compression applied by the first trigger 22 against the spring 28.

The spring 28 applies tension on the first trigger 22, maintaining the first trigger 22 in an inoperable position when not compressed. Since the spring 28 applies pressure on both the stable handgrip 10 and the first trigger 22, the tension on first trigger 22 is enough to facilitate smooth operation of the instrument 1.

When the first trigger 22 is fully released and rests again in a disengaged position, the safety apparatus 12 automatically locks, shutting power off to the drive system 80. The user can reposition the instrument 1 and start the process all over again.

Furthermore, the sensors 51 on the sides may indicate dangerous proximity of any nerve endings that may become positioned next to the rotating deburring burr bit 40, during operation. If a nerve ending is encountered, for any reason, during operation of the instrument, the sensors 51 would trigger lose power to the drive system 80, such that the drive system 80 stops immediately. Additionally, the sensors 51 would simultaneous alert the surgeon of a proximate nerve ending, through a warning tone.

It is also possible to have the sensor 51, positioned on the bottom of the tip portion 4, to provide a safety tone, such that the surgeon would be aware that the burr is in a safe position, and the nerve ending is directly below and opposite of the deburring bit 40.

A description of a second embodiment of the present invention will now be given.

It should be noted that the second embodiment to be described below is one in which a safety apparatus 12 and the tip portion 4 are different from the ones described above.

Hereinafter, in the drawings, the same components as those in the first embodiment are assigned the same reference numerals as those in the first embodiment and description thereof will be omitted, and only a difference from the first embodiment will be described.

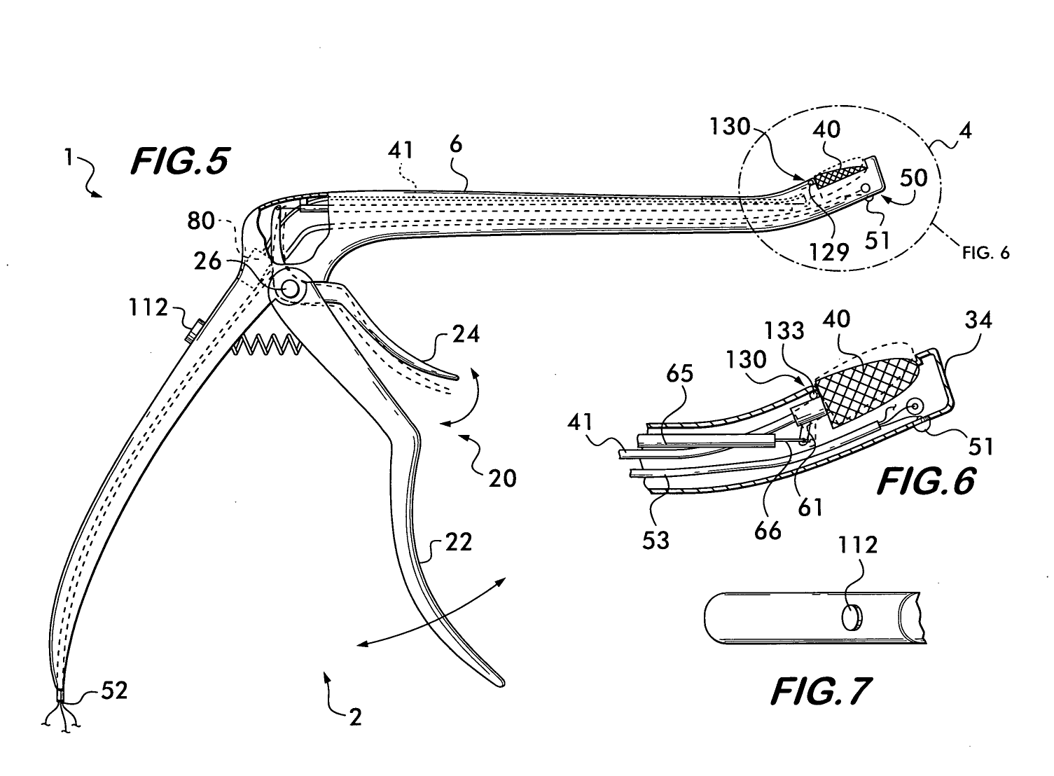

FIGS. 5 and 6 illustrate a second embodiment of the invention, wherein the tip portion 4 is provided as a unitary extension of the shaft 6. The angle θ between the tip portion 4 and the shaft 6 is fixed and determined prior to manufacturing. The design should accommodate to the type of surgery or user preference.

When the tip portion 4 and shaft 6 are prepared as a single piece, the second trigger 24 is prepared to adjust the bit 40, rather than the tip portion 4. According to the second embodiment, the bit 40 is pivotally connected to the tip portion 4 at a pivot point 130. A hollow axel rod 133 rigidly fastens to the top of the drive shaft 41. Two holes formed on each side of the tip portion 4 receive a locking pin 129, which also is received by the hollow axel rod 133, creating a pivot point 130. A lever 134, at one end, rigidly attaches to the underside of the drive shaft 41, while connecting to the telescoping actuator 64 at the other end. As discussed above, the telescoping actuator 64 connects to the proximal end of the second trigger 24. Such a construction, as illustrated in the second embodiment, allows the user to adjust the bit 41 by as much a 1-3 mm, exposing the top side of bit 40 from the inside of the tip portion 4.

In FIGS. 6 and 7, the safety apparatus 112 is an electronic button connecting to the drive system 80. The safety apparatus 112 is positioned on the handgrip 10 in such a way that the safety apparatus 112 may be functioned by use of a free thumb, and having the palm rest on the handgrip 10. It is possible, however, to include the safety apparatus 112 in other areas of the surgical instrument 1, that may be convenient for one hand use of the surgical instrument 1. Additionally, it is possible to mechanically connect the safety apparatus 112 to the first and second triggers 22, 24, through the handgrip 10, negating operation of either trigger 22, 24, until the safety apparatus 112 is unlocked.

The foregoing illustrates some of the possibilities for practicing the invention. Many other embodiments are possible within the scope and spirit of the invention. It is, therefore, intended that the foregoing description be regarded as illustrative rather than limiting, and that the scope of the invention is given by the appended claims together with their full range of equivalents.

What is claimed is:

1. A medical instrument, comprising:
a hand piece on a proximal end of a shaft and a hollow housing tip on a distal end of the shaft;
a hand grip positioned on a rear side of the hand piece;
a trigger portion positioned on front side of the hand piece;
the hand grip and the squeezable trigger portion positioned to mutually oppose each other, the trigger portion independently compressible of the handgrip;
a rotatable surgical tool bit controllable by the trigger portion, the trigger portion and bit both connected to a drive system;
a safety provided on the grip portion; and
a monitoring system disposed on the tip portion.

2. The medical instrument of claim 1 wherein the tip is rotatable at a pivot point.

3. The medical instrument of claim 2, wherein the tip is angled.

4. The medical instrument of claim 1, wherein the tip houses the bit.

5. The medical instrument of claim 1, wherein the trigger portion includes a first trigger and a second trigger.

6. The medical instrument of claim 5, wherein the first trigger controls the bit rotation rate.

7. The medical instrument of claim 5, wherein the second trigger controls the angle of the tip portion.

8. The medical instrument of claim 5, wherein the second trigger controls the height of the bit.

9. The medical instrument of claim 1, wherein the safety is a switch.

10. The medical instrument of claim 1, wherein the safety automatically locks after trigger portion is disengaged.

11. The medical instrument of claim 1, wherein the safety slides to unlock.

12. The medical instrument of claim 1, wherein the safety depresses to unlock.

13. The medical instrument of claim 1, wherein the rotatable surgical tool bit is bullet shaped.

14. The medical instrument of claim 1, wherein the rotatable surgical tool bit attaches to the drive shaft by a screw and thread fastening.

15. The medical instrument of claim 1, wherein the bit rotates by a higher rate with increased compression of the first trigger.

16. The medical instrument of claim 1, wherein the monitoring system includes at least one sensor positioned on the tip portion of the instrument, the sensor attaching to an external CPU through conductors, the external CPU used to identify signals transferred between a nerve and the sensor.

17. The medical instrument of claim 16, wherein the monitoring system includes 3 sensors, one sensor positioned on the underside of the tip portion and two sensors placed on each side of the tip portion.

18. The medical instrument of claim 17, wherein each sensor is independently connected to the external CPU.

19. The medical instrument of claim 16, wherein the sensor receives a signal.

20. The medical instrument of claim 16, wherein the sensor sends a signal.

21. A medical instrument monitoring system comprising two or more sensors positioned on sides of a distal end of a medical instrument, each sensor attaching to an external CPU through conductors, the external CPU used to identify signals transferred between a nerve and the sensor.

22. The medical instrument monitoring system of claim 21, wherein three sensors are positioned on a distal end of the medical instrument, one sensor positioned on the underside of the tip portion and two sensors placed on each side of the tip portion.

23. The medical instrument of claim 21, wherein each sensor is independently connected to the external CPU.

24. A method of positioning a rongeur in a surgery site comprising:
positioning a tip portion into an affected region;
monitoring signals from at least one sensor to further position into proximate surgery site;
adjusting a second trigger to position a deburring bit;
releasing a safety in order to operate rotation of the deburring bit;
compressing a first trigger to adjust rate of bit rotation;
releasing the first trigger completely which activates the safety.
